(19) United States
(12) Patent Application Publication
(10) Pub. No.: US 2015/0283239 A1
(43) Pub. Date: Oct. 8, 2015

(54) GRAPHENE DERIVATIVE-BASED COMPOSITION FOR DRUG DELIVERY AND PREPARATION METHOD THEREOF

(71) Applicant: Research & Business Foundation SUNGKYUNKWAN UNIVERSITY, Suwon-si (KR)

(72) Inventors: Hyoyoung LEE, Suwon-si (KR); Surajit SOME, Suwon-si (KR)

(73) Assignee: Research & Business Foundation SUNGKYUNKWAN UNIVERSITY, Suwon-si (KR)

(21) Appl. No.: 14/681,509

(22) Filed: Apr. 8, 2015

(30) Foreign Application Priority Data
Apr. 8, 2014 (KR) 10-2014-0042070

(51) Int. Cl. A61K 47/02; A61K 9/14; A61K 31/12

(52) U.S. Cl. CPC A61K 47/02; A61K 31/12; A61K 9/143

(57) ABSTRACT

A graphene derivative-based composition for drug delivery, a method of preparing the graphene derivative-based composition, and a method of drug delivery using a graphene derivative-based carrier are provided. The graphene derivative-based composition for drug delivery includes a nanocomposite including a drug loaded on a carrier.

### CROSS-REFERENCE TO RELATED APPLICATION

This application claims the benefit under 35 USC 119(a) of Korean Patent Application No. 10-2014-0042070 filed on Apr. 8, 2014, in the Korean Intellectual Property Office, the entire disclosures of which is incorporated herein by reference for all purposes.

### BACKGROUND

1. Field

The following description relates to drug delivery techniques, graphene derivative-based compositions for drug delivery, drug carriers and methods of preparing graphene derivative-based compositions for drug delivery applications.

2. Description of Related Art

With the recent rapid development of nano-biotechnology, the potential use of nanomaterials as carriers for drug delivery applications for cancer therapy has received much attention. Carbon nanostructures [e.g., graphene-derivatives and graphene quantum dots (GODS)] exhibit satisfactory biocompatibility, low toxicity, excellent physical properties, a surface amenable to modification, improved multifunctionality, and compatibility with conventional graphene technology [F. Peng, Y. Su, X. Wei, Y. Lu, Y. Zhou, Y. Zhong, S. Lee, Y. He, Aνγεω. Xηεμ. Iντ. Eδ. 2013, 52, 1457; Z. Liu, J. T. Robinson, X. M. Sun, H. J. Dai, [symbol] . Aμ. Xηεμ. Σoχ. 2008, 130, 10876].

In particular, the use of graphene-derivatives is very promising for a wide range of biological applications, including the recent development of GQD-based bioprobes for tumor imaging [M. Nurunnabi, Z. Khatun, K. M. Huh, S. Y. Park, D. Y. Lee, K. J. Cho, Y. Lee, AXΣ Nανo 2013, 7, 6858; A. Nahain, J. Lee, I. In, H. Lee, K. D. Lee, J. H. Jeong, S. Y. Park, Moλ. πηαρμαχεντιχσ 2013, 10, 3736]. However, reported drug deliveries (including proteins, amphiphilic block copolymers, lipids, and inorganic nanoassemblies) have drawbacks including premature drug release due to their limited stability.

Recently, it has been demonstrated that graphene materials can be loaded on aromatic ring-containing anticancer drugs such as doxorubicin (DOX) and camptothecin (CPT) with ultrahigh efficiency. It has been reported that graphene-oxide-derivative-Cur (Curcumin) composites also have anticancer activity, but they are neither very effective nor easily prepared. Their use has not been demonstrated in actual applications, i.e., treatment of tumors or inhibition of tumor proliferation, and their drug loading capability is low, suggesting that they are incomplete for clinical applications. In addition, there have been reports of graphene quantum dot-loaded drugs, but it has been demonstrated that their efficiency is insufficient, and they are not useful in actual applications such as tumor treatment [Z. Wang, J. Xia, C. Zhou, B. Via, Y. Xia, F. Zhang, Y. Li, L. Xia, J. Tang, Xoλλoιδσ ανδ Σνρφαχεσ B: Bιoιντερφαχεσ 2013, 112, 192; C. Wang, C. Wu, X. Zhou, T. Han, X. Xin, J. Wu, J. Zhang, S. Guo, Σχι Pεπ. 2013, 3, 2852].

Moreover, drug ingredients can be easily removed by renal clearance and distribution into non-targeted tissues, and thereby, causing an insufficient drug concentration at a tumor site and restricting effects in therapy. The use of graphene-based nanomaterials deserves attention for overcoming a physiological barrier because the nanomaterials have a property of excellent absorption in bloodstream. Thus, a graphene-based drug delivery nano-system that has compatibility with a physiological environment is desirable. Of particular interest, recent studies have demonstrated that a graphene derivative exhibits excellent catalytic performance owing to its large surface area to generate polar interaction with an oxygen-containing functional group, which is an essential factor for enhancement of drug-loading capacity [Z. Liu, J. T. Robinson, X. M. Sun, H. J. Dai, [symbol] . Aμ. Xηεμ. Σoχ. 2008, 130, 10876; L. M. Zhang, J. G. Xia, Q. H. Zhao, L. W. Liu, Z. J. Zhang, Σμαλλ 2010, 6, 537; J. Wu, Y. Wang, X. Yang, Y. Liu, J. Yang, R. Yang, N. Zhang, Nανoτεχηνoλoγψ 2012, 23, 355101].

### SUMMARY

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

In view of the foregoing issues, the present disclosure provide a graphene derivative-based composition for drug delivery, which includes a graphene oxide (GO) prepared by loading various drugs thereon, a double oxidized graphene oxide (DGO), and graphene quantum dots (GQDs), and a method of preparing the graphene derivative-based composition.

However, issues resolved by the present disclosure are not limited to those described above. Although not described herein, other issues resolved by the present disclosure can be clearly understood by those skilled in the art from the following description.

In a first aspect, a graphene derivative-based composition includes a nanocomposite including a drug loaded on a carrier comprising a graphene derivative.

The graphene derivative may include a graphene oxide, a double-oxidized graphene oxide, or a graphene quantum dot.

The drug may include a drug for a treating or a preventing of a disease selected from the group consisting of a cancer, a HIV infection, and a neurological disease, a cardiovascular disease, and a skin disease.

The drug may include an antitumor agent.

The antitumor agent may include a curcumin, a doxorubicin, a paclitaxel, an etoposide, a vinca alkaloid, a vinblastine, or a colchicine.

The drug may have a nanoparticle shape.

The graphene derivative may have a relatively high content of oxygen-containing functional group.

A released amount of the drug loaded on the carrier may be controlled depending on a pH.

The pH may be controlled to an acidic, a neutral, and an alkaline range.

A size of a particle of the nanocomposite may range between about 50 nm and about 5,000 nm

The carrier comprises a graphene quantum dot, and the carrier is configured to deliver drug or to be used as a bioprobe for a cell imaging using a luminescent property of the graphene quantum dot.

In another general aspect, a pharmaceutical composition includes the graphene derivative-based composition described above.

In yet another general aspect, a method of preparing a graphene derivative-based composition involves adding a drug to a dispersion comprising a carrier comprising a graphene derivative to form a graphene derivative-drug nanocomposite in which the drug is loaded on the carrier.

The dispersion may include water as a solvent.

A pH of the dispersion to which the carrier is added may be controlled to an alkaline range.

The graphene derivative may comprise a graphene oxide, a double oxidized graphene oxide, or a graphene quantum dot.

The drug may include a drug for therapy or prevention of a disease selected from the group consisting of a cancer, a HIV infection, and a neurological disease, a cardiovascular disease, and a skin disease.

The antitumor agent may comprise a curcumin, a doxorubicin, a paclitaxel, an etoposide, a vinca alkaloid, a vinblastine, or a colchicine.

The drug may have a nanoparticle shape.

The graphene derivative may have a relatively high content of oxygen-containing functional group.

In accordance with the example embodiments, it is possible to prepare a graphene derivative-based composition for drug delivery, which includes drug for treating or preventing of cancers or various disorders. Considering that the graphene derivative can be easily prepared with a relatively high yield rate and low costs, the graphene-based nano-carrier serves as a strong realizable means for cancer therapy, and can be used as a material competing with or supplementing nanomaterial-based delivery materials in the art of the present disclosure.

Especially, a high-performance drug delivery composition can be prepared by using a graphene derivative having a property of super-high drug-loading capacity for delivery of anticancer drug (Curcumin), and the drug delivery composition can be used for superior synergistic therapy of cancer cells in vitro and in vivo, and simultaneously, can be used as a superficial bioprobe for tumor imaging, providing a new opportunity for biological and medical applications.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

FIG. 1 is a schematic diagram of a method of preparing various Curcumin-graphene composites (GO-Cur, DGO-Cur, and GQD-Cur) and their relative anticancer effects according to an example embodiment of the present disclosure.

FIG. 2A illustrates FTIR spectra of GO, DGO, GQD, GO-Cur, DGO-Cur and GQD-Cur composites and Curcumin prepared according to an example of the present disclosure.

FIG. 2B illustrates UV-vis spectra of GO, DGO, GQD, GO-Cur, DGO-Cur and GQD-Cur composites and Curcumins prepared according to an example of the present disclosure.

FIG. 3 shows SEM and TEM images of different materials in an Example of the present disclosure: (a) SEM image of GO, (b) SEM image of DGO, (c) SEM image of GQD, (d) SEM image of Curcumin, (e) SEM image of a GO-Cur composite, (f) SEM image of a DGO-Cur composite, (g) SEM images of a GQD-Cur composite, (h) TEM images of GQD, and (i) TEM images of the GQD-Cur composite.

FIGS. 4A to 4E are graphs showing behaviors of graphene-derivatives in an Example of the present disclosure. FIG. 4A is a graph showing photoluminescence (PL) intensity of GQDs at a 512 nm wavelength of graphene-derivatives in an Example of the present disclosure. FIG. 4B is a graph showing PL intensities of GQDs and Curcumin-loaded GQDs of graphene-derivatives in an Example of the present disclosure. FIG. 4C is a graph showing amounts of Curcumin loaded at different pH values (pH 5, pH 7.5, and pH 9) and in various Curcumin concentrations according to an Example of the present disclosure. FIG. 4D is a graph showing amounts of Curcumin loaded on GO, DGO, and GQD under pH control (pH 5, pH 7.5, and pH 9) according to time according to an Example of the present disclosure. FIG. 4E is a graph showing In vitro concentration-dependent cell viability of HCT116 cells, wherein cells were incubated with free GO, DGO, GQD, GO-Cur, DGO-Cur, GQD-Cur and free Cur for 24 hours as described above.

FIG. 5 shows typical nuclear morphology images after DAPI staining and images showing analysis by a fluorescence microscope, in an Example of the present disclosure. The images were obtained after treating HCT116 cells by using (a) PBS, (b) GO, (c) DGO, (d) GQD, (e) Cur, (f) GO-Cur, (g) DGO-Cur, and (h) GQD-Cur.

FIG. 6A to 6E show in vivo experimental data in an Example of the present disclosure. FIG. 6A is a graph that compares tumor volumes of mice (n=6) treated with PBS, DGO, GQD, DGO-Cur, GQD-Cur and Cur according to an Example of the present disclosure. FIG. 6B is a graph that compares tumor weights of mice (n=6) treated with PBS, DGO, GQD, DGO-Cur, GQD-Cur and Cur according to an Example of the present disclosure. FIG. 6C illustrates photographs of mice (n=6) after 14 days from treatment with PBS, DGO, GQD, DGO-Cur, GQD-Cur and Cur after 14 days according to an Example of the present disclosure. FIG. 6D illustrates photographs of tumors of mice (n=6) after 14 days from treatment with PBS, DGO, GQD, DGO-Cur, GQD-Cur and Cur according to an Example of the present disclosure. FIG. 6E illustrates in vivo images of tumor-bearing mice after injection of GQDs and GQD-Cur (10 mg/kg) according to an Example of the present disclosure.

FIG. 7 shows XPS spectra of GO, DGO and GQD in an Example of the present disclosure.

FIG. 8A is a graph showing size distribution of GQDs according to an Example of the present disclosure.

FIG. 8B is an image of GQDs under UV light at 365 nm excitation, according to an Example of the present disclosure.

FIG. 9 is a graph showing PL intensity of Curcumin in an Example of the present disclosure. The Curcumin is initial Cur dissolved in DMSO and diluted in deionized water.

FIG. 10 is an AFM image of GQDs in an Example of the present disclosure.

FIG. 11 is a graph showing weights of differently-treated mice, in an Example of the present disclosure.

FIG. 12 shows images of organs of mice treated with DGO, GQD, DGO-Cur, GQD-Cur, Cur, and PBS (treated as a control sample), in an Example of the present disclosure.

FIG. 13 shows images of histological analysis of tissues from mice (heart) treated with DGO, GQD, DGO-Cur, GQD-Cur, Cur, and PBS (treated as a control sample), in an Example of the present disclosure.

FIG. 14 shows images of histological analysis of tissues from mice (kidney) treated with DGO, GQD, DGO-Cur, GQD-Cur, Cur, and PBS (treated as a control sample), in an Example of the present disclosure.

FIG. 15 shows images of histological analysis of tissues from mice (liver) treated with DGO, GQD, DGO-Cur, GQD-Cur, Cur, and PBS (treated as a control sample), in an Example of the present disclosure.

FIG. 16 shows images of histological analysis of tissues from mice (lung) treated with DGO, GQD, DGO-Cur, GQD-Cur, Cur, and PBS (treated as a control sample), in an Example of the present disclosure.

FIG. 17 shows images of histological analysis of tissues from mice (spleen) treated with DGO, GQD, DGO-Cur, GQD-Cur, Cur, and PBS (treated as a control sample), in an Example of the present disclosure.

### DETAILED DESCRIPTION

The following detailed description is provided to assist the reader in gaining a comprehensive understanding of the methods, apparatuses, and/or systems described herein. However, various changes, modifications, and equivalents of the methods, apparatuses, and/or methods described herein will be apparent to one of ordinary skill in the art. For example, the sequences of operations described herein are merely examples, and are not limited to those set forth herein, but may be changed as will be apparent to one of ordinary skill in the art, with the exception of operations necessarily occurring in a certain order. Also, descriptions of functions and constructions that are well known to one of ordinary skill in the art may be omitted for increased clarity and conciseness.

Throughout the drawings and the detailed description, the same reference numerals refer to the same elements. The drawings may not be to scale, and the relative size, proportions, and depiction of elements in the drawings may be exaggerated for clarity, illustration, and convenience.

The features described herein may be embodied in different forms, and are not to be construed as being limited to the examples described herein. Rather, the examples described herein have been provided so that this disclosure will be thorough and complete, and will convey the full scope of the disclosure to one of ordinary skill in the art.

Throughout the whole document of the present disclosure, the terms “connected to” or “coupled to” are used to designate a connection or coupling of one element to another element and include both a case where an element is “directly connected or coupled to” another element and a case where an element is “electronically connected or coupled to” another element via still another element.

Throughout the whole document of the present disclosure, the term “on” that is used to designate a position of one element with respect to another element includes both a case that the one element is adjacent to the another element and a case that any other element exists between these two elements.

Throughout the whole document of the present disclosure, the term “comprises or includes” and/or “comprising or including” means that one or more other components, steps, operations, and/or the existence or addition of elements are not excluded in addition to the described components, steps, operations and/or elements. As used throughout the document of the present disclosure, the terms “about or approximately” or “substantially” are intended to have meanings close to numerical values or ranges specified with an allowable error and intended to prevent accurate or absolute numerical values disclosed for understanding of the present invention from being illegally or unfairly used by any unconscionable third party. As used throughout the document of the present disclosure, the term “step of” does not mean “step for.”

Throughout the whole document of the present disclosure, the term “combinations of” included in Markush type description means mixture or combinations of one or more components, steps, operations and/or elements selected from a group consisting of components, steps, operation and/or elements described in Markush type and thereby means that the disclosure includes one or more components, steps, operations and/or elements selected from the Markush group.

Throughout the whole document of the present disclosure, the description “A and/or B” means “A or B, or A and B.”

Hereinafter, example embodiments and Examples of the present disclosure are described in detail with reference to the accompanying drawings. However, the present disclosure may not be limited to the example embodiments, the Examples, and the drawings.

The first aspect of the present disclosure provides a graphene derivative-based composition for drug delivery, which contains a nano-composite including drug loaded on a carrier including a graphene derivative.

In accordance with an example embodiment of the present disclosure, the graphene derivative may include a graphene oxide (GO), a double oxidized graphene oxide (DGO), or graphene quantum dots (GODS), but not be limited thereto.

In accordance with an example embodiment of the present disclosure, the drug may include drug for treating or preventing of a disease selected from the group consisting of a cancers, a HIV infection, a neurological diseases, a cardiovascular diseases, and a skin diseases, but not be limited thereto.

In accordance with an example embodiment of the present disclosure, the drug may include antitumor agent, but not be limited thereto.

In accordance with an example embodiment of the present disclosure, the antitumor agent may include a Curcumin, a doxorubicin, a paclitaxel, an etoposide, a vinca alkaloid, a vinblastine, or a colchicine, but not be limited thereto.

In accordance with an example embodiment of the present disclosure, the drug may have a nanoparticle shape, for example, when it includes anticancer drug, but not be limited thereto.

In accordance with an example embodiment of the present disclosure, the graphene derivative may have a relatively high content of an oxygen-containing functional group, but not be limited thereto.

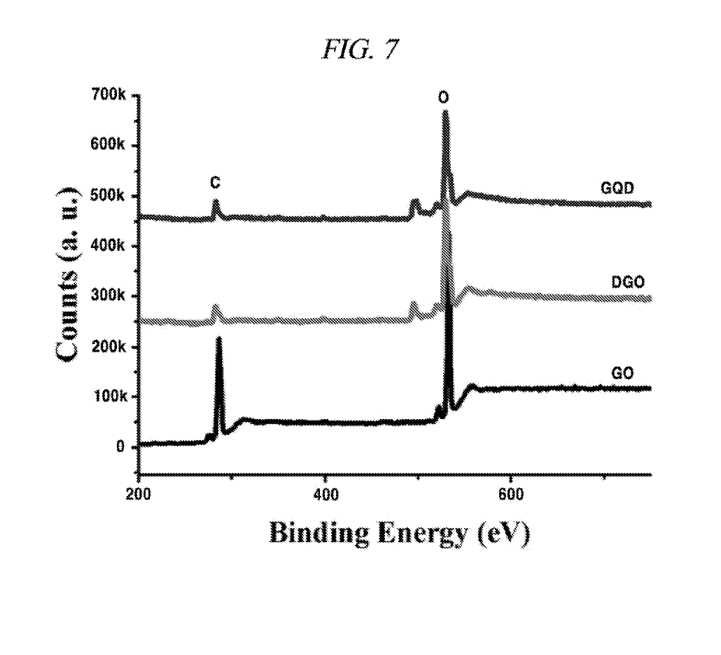

As shown in FIG. 7, the prepared graphene derivative including GO, DGO, and GQDs exhibited a low carbon/oxygen (C/O) ratio of about 2.2 or about 1, which may imply that an oxygen amount of nano-sheets of the graphene derivative increases, and the graphene derivative includes a large amount of the oxygen-containing functional group, but the present disclosure may not be limited thereto.

In accordance with an example embodiment of the present disclosure, a released amount of the drug loaded on the carrier may be controlled depending on a pH, but not be limited thereto.

In accordance with an example embodiment of the present disclosure, the pH may be controlled in an acidic, neutral and alkaline range, but not be limited thereto.

In accordance with an example embodiment of the present disclosure, a size of a particle of the nanocomposite may be from about 50 nm to about 5,000 nm, but not be limited thereto. For example, the size of the carrier particles may be from about 50 nm to about 5,000 nm, from about 100 nm to about 5,000 nm, from about 500 nm to about 5,000 nm, from about 1,000 nm to about 5,000 nm, from about 1,500 nm to about 5,000 nm, from about 2,000 nm to about 5,000 nm, from about 2,500 nm to about 5,000 nm, from about 3,000 nm to about 5,000 nm, from about 3,500 nm to about 5,000 nm, from about 4,000 nm to about 5,000 nm, from about 4,500 nm to about 5,000 nm, from about 50 nm to about 4,500 nm, from about 50 nm to about 4,000 nm, from about 50 nm to about 3,500 nm, from about 50 nm to about 3,000 nm, from about 50 nm to about 2,500 nm, from about 50 nm to about 2,000 nm, from about 50 nm to about 1,500 nm, from about 50 nm to about 1,000 nm, from about 50 nm to about 500 nm, or from about 50 nm to about 100 nm, but not be limited thereto.

In accordance with an example embodiment of the present disclosure, the carrier containing a graphene quantum dot may be used for drug delivery composition, or used as a bioprobe for a cell imaging using a luminescent property of the graphene quantum dot, but not be limited thereto.

The second aspect of the present disclosure provides a pharmaceutical composition, which includes the graphene derivative-based drug delivery composition prepared according to the first aspect of the example embodiments.

All the descriptions of the first aspect of the present disclosure are applied to the second aspect of the present disclosure.

The third aspect of the present disclosure provides a preparing method of a graphene derivative-based drug delivery composition, which includes adding drug to a dispersion including a carrier containing a graphene derivative to form a graphene derivative-drug nanocomposite in which the drug is loaded on the carrier.

In accordance with an example embodiment of the present disclosure, the dispersion may include water as a solvent, but not be limited thereto.

In accordance with an example embodiment of the present disclosure, a pH of the dispersion including the carrier containing the graphene derivative may be controlled in an alkaline range, but not be limited thereto.

In accordance with an example embodiment of the present disclosure, the graphene derivative may include a graphene oxide, a double oxidized graphene oxide, or graphene quantum dots, but not be limited thereto.

In accordance with an example embodiment of the present disclosure, the drug may include drug for treating or preventing of a disease selected from the group consisting of a cancer, a HIV infection, a neurological diseases, a cardiovascular diseases, and a skin diseases, but not be limited thereto.

In accordance with an example embodiment of the present disclosure, the drug may include antitumor agent including a Curcumin, a doxorubicin, a paclitaxel, an etoposide, a vinca alkaloid, a vinblastine, or a colchicine, but not be limited thereto.

In accordance with an example embodiment of the present disclosure, the drug may have a nanoparticle shape, for example, when it includes antitumor agent, but not be limited thereto.

In accordance with an example embodiment of the present disclosure, the graphene derivative may have a relatively high content of an oxygen-containing functional group, but not be limited thereto.

Hereinafter, Examples of the present disclosure are described in detail. However, the present disclosure may not be limited to the Examples.

### EXAMPLES

### <Materials>

Natural graphite (Bay Carbon, SP-1 graphite), sulfuric acid (95% to 97%), hydrogen peroxide (30 wt. %), potassium permanganate, sodium nitrate, sodium hydroxide, citric acid, and Curcumin were purchased from commercial sources and used as they were.

### <Preparation of Graphene Oxide (GO)>

GO sheets were prepared from natural graphite powders by using the Hummer's method modified by using sulfuric acid, potassium permanganate, and sodium nitrite.

### <Synthesis of Doubled-Oxidized Graphene Oxide (DGO)>

DGO sheets were synthesized from the above-prepared GO through a conventionally reported process.

### <Synthesis of Graphene Quantum Dots (GQDs)>

More oxygen-containing GQDs were synthesized by a conventionally reported process.

### <Synthesis of GO-Cur, DGO-Cur, and GQD-Cur Composites>

GO, DGO, and GQD nano-sheets were dispersed in deionized water (about 200 μg/mL), the aqueous solution was adjusted to about pH 9, and finally, Cur w.r.f (with respect to) in an amount one (1) to five (5) times GO, DGO, and GQD was mixed with the aqueous solution. The reaction mixture was stirred at 4° C. for 30 minutes, followed by centrifugation at 14,000 rpm for 20 minutes and washing three (3) times with deionized water, and the resulting pellet was dried under vacuum. However, GQD-Cur was collected by recrystallization of unattached Cur and evaporation of the resulting solution.

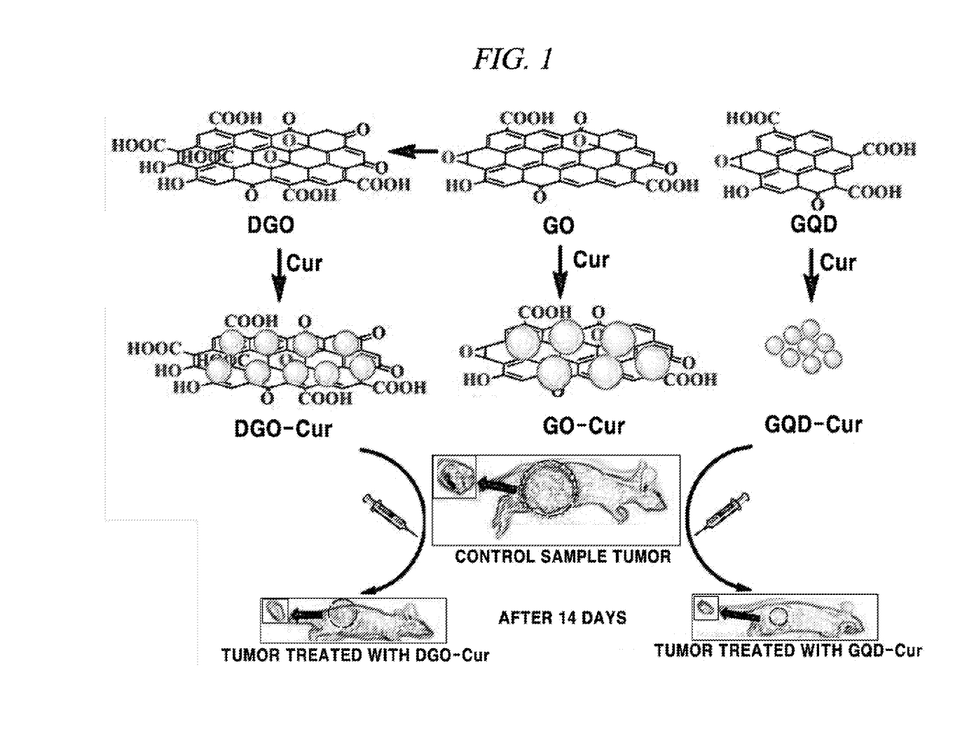

Herein, DGO-Cur and GQD-Cur refers to DGO and GDQ molecules loaded with Curcumin, as illustrated in FIG. 1. A loading may occur via a covalent bond interaction between the carried substance, such as Curcumin, and the DGO and/or GDO molecules, or via non-covalent interactions such as Van der Waals forces, electrostatic interaction, hydrogen bonding, a π-π stacking, ionic forces, hydrophobic and hydrophilic interaction, and the like.

### <Drug Loading Capacity Calculation>

Drug loading capacity=(W_{initial Cur}−W_{Cur in excess})/(W_{graphene-derivative})(Mg/g)², where W_{initial Cur} is the initial weight of Cur added, W_{Cur in excess} is the weight of Cur in the supernant, and W_{graphene-derivative} is the weight of graphene derivatives (GO, DGO, and GQD). The weight in excess of Cur was 184 μg/mL at pH 9 in loading where Cur concentration=1,000 μg/mL, and thus, the weight of Cur loaded on GQD was 816 μg. As a result, the loading capacity of Cur on GQD corresponded to 40,800 mg/g, and when Cur is 1 mg, Curs loaded on GO and DGO were 20,800 mg/g and 38,800 mg/g, respectively, at pH 9. Graphene derivative-Cur composites prepared at the pH 9 condition were re-dispersed in deionized water at different pH values (5, 7.5, and 9) for different times (5, 10, 15, 20 and 24 hours) at a room temperature. Cur molecules that remained on the graphene derivative surfaces were calculated based on the weight of released Cur.

### <Characterization>

All X-ray photoelectron spectroscopy (XPS) measures were performed at 100 W by using a solid-color Al-K X-ray source by means of SIGMA PROBE (Thermo, U.K.). FR-IR spectra were performed by using the Thermo Nicolet AVATAR 320 machine. Microstructures were detected by a field emission scanning electron microscope (FE-SEM; JSM-6701F/INCA Energy, JEOL). All UV-vis absorption spectra were recorded by using a double-beam UV-1650PC spectrophotometer (Shimadzu). The atomic force microscope (AFM) was measured by using the SPA400 equipment having the SPI-3800 controller (Seiko Instrument Industry Co.). The TEM images were measured by JEOL JEM 3010. Photoluminescence excitation and release were measured by the luminescence analyzer, Fluoro Mate FS-2 (Scinco, Korea).

### <Cell Culture>

Human colon cancer cells (HCT116) were maintained in a Dulbecco's modified Eagle medium (DMEM), and supplemented with 10% fetal bovine serum (FBS) and antibiotics (10,000 μg/mL streptomycin and 10,000 unit/mL penicillin) at 37° C. in a humidified atmosphere containing 5% CO₂(v/v).

### <Cytotoxicity Evaluation>

In vitro cytotoxicity was measured by using standard colorimetric 3-(4,5-dimethylthiazol-2-yl)-2,5-diphenyl tetrazolium bromide (MTT) analysis. For the MTT analysis, HCT116 cells were seeded in a 96-well cell-culture plate at 1×10⁴/well, and cultured for 12 hours at 37° C. under 5% CO₂. Thereafter, the HCT116 cells were cultured for 24 hours by using various concentrations (6.125, 12.5, 25, 50, and 100 μg/mL) of GO, DGO, GQD, Cur, GO-Cur, DGO-Cur, and GQD-Cur. Next, 20 μl stock MTT (5 mg/mL) was added to each of the cells, and the cells were cultured under 5% CO₂ at 37° C. for 4 hours. After the culture for additional 4 hours, the resulting formazan crystals were dissolved in dimethyl sulfoxide (DMED, 100 μl), and absorbance intensity was measured by a microplate reader at 570 nm. All the experiments were conducted in quadruplicate, and the relative cell viability (%) was expressed as a percentage relative to untreated control cells.

### <Immunofluorescence>

For 4,6-diamidino-2-phenylindole (DAPI) staining, the cells were proliferated on a cover glass until 80% fusion was reached. Thereafter the cells were then washed with PBS, placed in methanol (about 20° C.) for 2 minutes, and stained with 1 mM DAPI in the dark for 5 minutes. After washing with PBS, nuclear morphology was analyzed by a fluorescence microscopy.

### <In Vivo Drug Delivery>

Six (6) to seven (7)-week-old Balb/c female nude mice (weight of 21 g to 25 g) were purchased from Orient Bio Inc., (Seoul, Korea), and were maintained in a 12-hour light/12-hour darkness cycle (12:12LD) at a temperature of 22° C. to 24° C., in humidity of 40% to 60%, and with food and water ad libitum. HCT116 was trypsinized, washed twice with serum-free DMEM, and dispersed at a density of 1×10⁷ cells/mL PBS. 100 mL of the dispersed cells was subcutaneously injected into the right back region of the mice. After the tumor size reached about 150 mm³, the mice were randomly divided into six (6) groups depending on differences in the minimum weight and tumor size. The mice were intratumorally injected with physiological saline, DGO, GQD, Cur, DGO-Cur, and GQD-Cur dispersions at a total dose of 10 mg/kg. The tumor size of each of the groups was measured by a caliper, and the tumor volume was calculated by using the following equation: tumor volume=ab²×0.5236, where ‘a’ is the maximum diameter of tumor, and ‘b’ is the minimum diameter of tumor. Relative tumor volumes were calculated as V/V₀ (V₀ was the tumor volume when the treatment was initiated).

### <Noninvasive Optical Imaging Study>

After 14 days, the tumor-transplanted mice were intratumorally injected with 10 mg/kg of the composites. The mice were anesthetized with ketamine (87 mg/kg) and xylazine (13 mg/kg) through intraperitoneal (IP) injection. Noninvasive images of the mice injected with GQD-Cur, GQD, and PBS were taken by an optical tomography system. The mice were placed in an imaging platform, and images were taken at 4-hour post-injection. The 3D scanning region of interest was selected by using a bottom-view charge-coupled device (CCD). All the images were taken by using the Optix in vivo imaging system (Optix MX3, ART Advanced Research Technologies INC, Canada).

### Histological Analysis>

For histological studies, the mice were tested for 2 weeks after the injection. Tissues (heart, liver, spleen, kidney, and lung) were collected from each of the groups, fixed in 10% formalin and embedded in paraffin. A multiple number of 4 μm-thick microtome sections from the tissues were stained with hematoxylin and eosin (H & E). The histological sections were detected under an optical microscope.

The inventors of the present disclosure describe an example for effective use of the graphene derivative-based drug nano-composites for cancer therapy by using a graphene oxide (GO), a double oxidized graphene oxide (DGO), and GQDs as novel nanovectors for delivery of the anticancer drug, Cur (FIG. 1).

The graphene derivative-Cur composites were quickly prepared by the new, simple, easy method, in which Cur was effectively attached to the surfaces of the graphene derivatives. It was hypothesized that increase in the number of oxygen-containing functional groups on the surfaces of the graphene derivatives should lead to increased attachment of Cur, thereby, producing the DGO-Cur composite having increased anticancer activity. In addition, GQDs should produce GQD-Cur with very high anticancer activity; where the large amount of Cur is due to the presence of the large surface area having oxygen-containing functional groups.

Unusually, the experiments conducted both in vitro and in vivo demonstrated that the graphene derivative-based nano-composites were highly effective for cancer therapy. Among the graphene derivatives, GQD can prepare the composite capable of carrying a large amount of Cur drug and serving as a bioprobe for tumor imaging.

Remarkably, GQDs have a maximum-efficiency drug-loading property of about 40,800 mg/g, which is the highest value ever reported for nanomaterial-based carriers.

### Example 1

### Characterization of the Graphene Derivative-Based Composition for Drug Delivery

Based on XPS analysis, the above-prepared GO had a low C/O ratio (2.2) (refer to FIG. 7). The resulting DGO exhibited a low C/O ratio of about 1, which corresponds to increase in the oxygen content of the DGO nano-sheets [B. J. Hong, O. C. Compton, Z. An, I. Eryazici, S. T. Nguyen, AXΣ Nανo 2012, 1, 63]. The above-prepared GQDs exhibited the low C/O ratio (refer to FIG. 7), but this implies that GQDs also include a large amount of oxygen-containing functional groups. Finally, a Cur-loaded composite for each of GO, DGO and GQD was prepared.

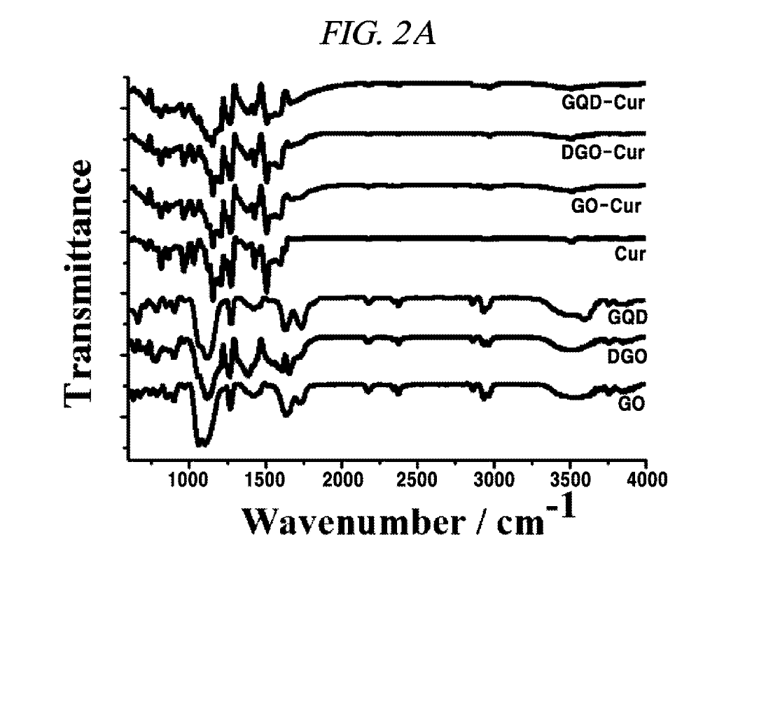

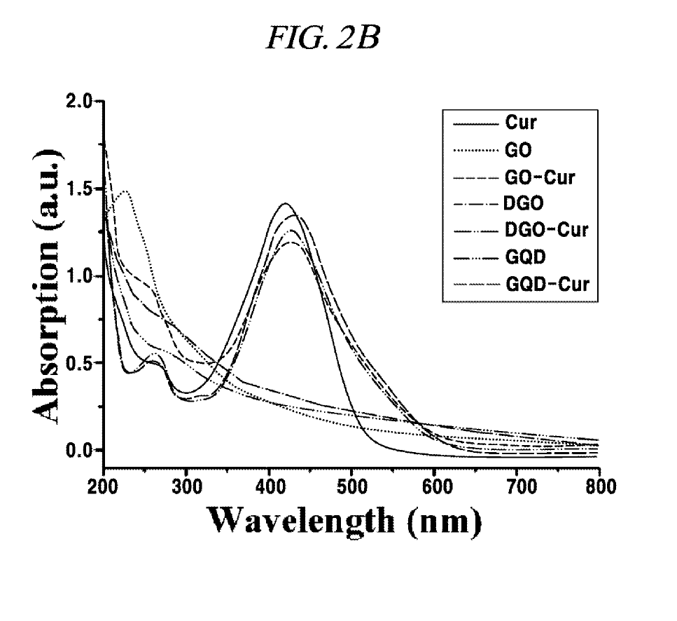

These materials were characterized by Fourier transform infrared spectroscopy (FT-IR), UV-vis absorption spectrum, and scanning electron microscopy (SEM) analysis. By using infrared spectrometry (FIG. 2A), characteristic absorption of different Cur functional groups on the graphene derivatives was analyzed. The presence of Cur physically attached to the graphene derivatives through polar interaction was confirmed by FT-IR.

The spectra of FIG. 2A show different types of oxygen functionalities in GO at 3,530 cm⁻¹ (o—H stretching vibrations), and different types of oxygen functional groups of GO at 1,729 cm⁻¹ (C═O stretching vibrations), 1,634 cm⁻¹ (C═C skeletal vibrations from unoxidized graphitic diamonds), and 1,058 cm⁻¹ (C—O stretching vibrations).

After the DGO derivative was produced by double oxidation of GO, the peak ratios of 3,529 cm⁻¹ and 1,737 cm⁻¹ (O—H stretching vibrations) with 1,116 cm⁻¹ (aromatic C—O stretching vibration) decreased, indicating that the GOs were functionalized by more oxygen-containing functional groups.

The spectra of FIG. 2A show the presence of C═C, C—O, C═O, and COOH bonds, which indicate that GQDs were functionalized by hydroxyl, carbonyl, and carboxylic acid groups [Z. Wang, J. Xia, C. Zhou, B. Via, Y. Xia, F. Zhang, Y. Li, L. Xia, J. Tang, Colloids and Surfaces B: Biointerfaces 2013,112, 192]. Cur exhibited remarkable peaks at 3,510 cm⁻, 1,510 cm⁻¹, 1,279 cm⁻¹, 1,152 cm⁻¹, and 959 cm⁻¹ (FIG. 2A) caused by stretching vibrations of OH, C—O, C—H, aromatic C—O, and C—O—C [P. R. K. Mohan, G. Sreelakshmi, C. V. Muraleedharan, R. Joseph, ζιβ. ΣπεχτρOσχ. 2012, 62, 77]. The GO-Cur composite exhibited characteristic Cur absorption features at 1,509 cm⁻, 1,272 cm⁻¹, and 1,153 cm⁻¹, which was very similar to that when Cur alone is used. The other composites (DGO-Cur and GQD-Cur) also exhibited characteristic peaks at 1,509 cm⁻¹, 1,275 cm⁻¹, and 1,154 cm⁻¹, which imply that they include Cur.

The intensity of the O—H stretching vibrations of all the composites decreased, indicating that Cur was successfully grafted onto GO, DGO and GQD.

The main absorption peak of GO appeared at 226.5 nm of the UV-vis spectrum (FIG. 2B). In case of using Cur alone, the absorption peak appeared at 419.2 nm, whereas the absorption peaks for DGO and GQD appeared at 286 nm and 292 nm, respectively. After the formation of the Cur composite with GO, DGO and GQD, red shift relative to the Cur peaks was detected. The main absorption peaks of GO-Cur, DGO-Cur, and GQD-Cur appeared at about 430 nm of the UV-vis spectrum, which correspond to about 10 nm red shift compared to the peaks when Cur exists alone, indicating the formation of composites.

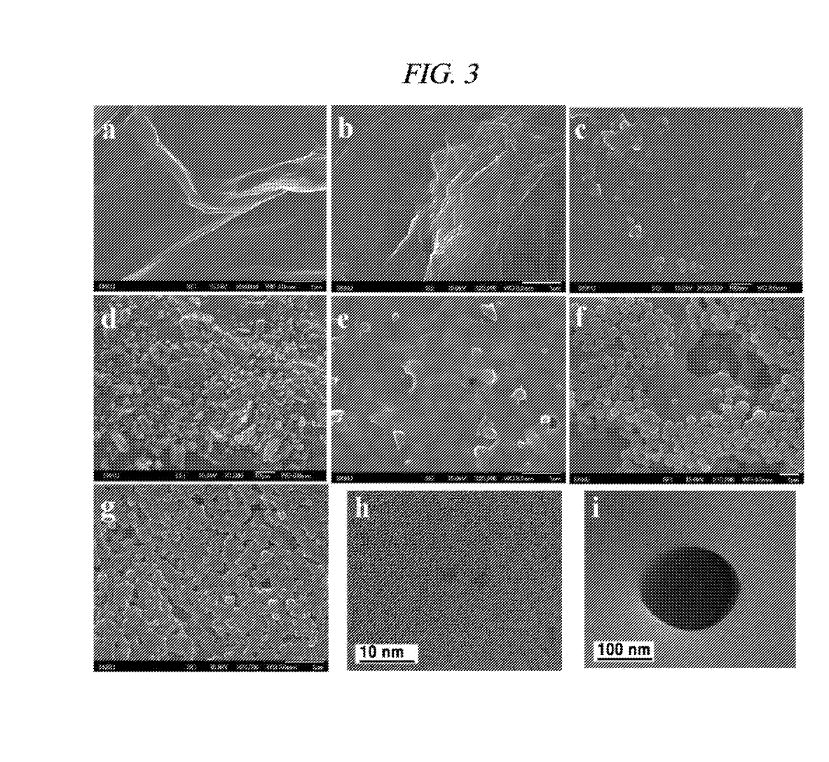

SEM analysis was used to determine surface morphologies of the various graphene derivatives (FIG. 3). A thin and wrinkled GO sheet was observed by using an SEM image of GO in (a) of FIG. 3. As shown in the SEM image of GO-Cur, Cur was physically attached to the surface of GO. The SEM image of the GO-Cur composite shows formation of Cur molecules on the wrinkled GO sheet in (e) of FIG. 3. An average size of the Cur nano-particles was about 150 nm. A DGO sheet was formed after another oxidization of the above-prepared GO in (b) of FIG. 3. The SEM image of DGO shows more wrinkled morphology compared to when GO exists alone. The SEM image of the DGO-Cur composite in (f) of FIG. 3 shows that the DGO surface has more Cur nano-particles than those of GO-Cur illustrated in (e) of FIG. 3. The average size of the Cur nano-particles was about 150 nm to about 120 nm.

FIG. 3 illustrates an SEM image of GQD in (c). The SEM image of the GQD-Cur composite in (g) of FIG. 3 shows that numerous round-shaped composites are included, and their average size is about 100 nm, whereas image (d) of FIG. 3 shows an SEM image of Cur alone. Thus, the SEM images verify the formation of the composites.

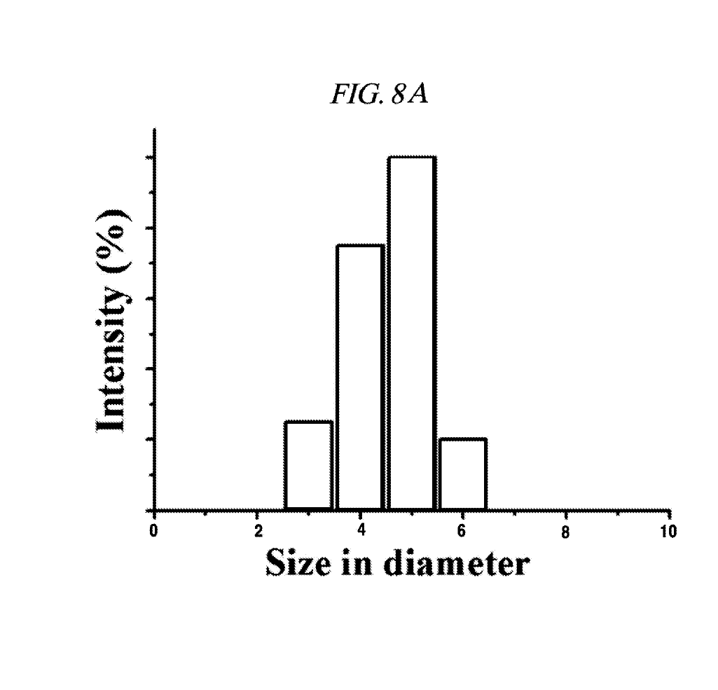

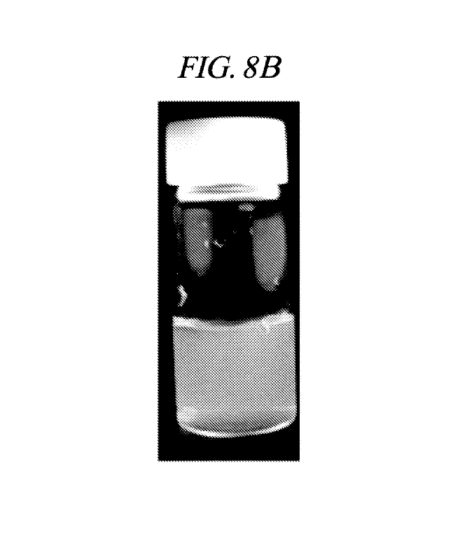

The HRTEM image (h) of FIG. 3 shows that synthesized GQDs have favorable shapes. As measured by HRTEM, the size and morphology analysis indicated that nano-sized GQDs have an average size (diameter) of 3 nm to 6 nm (refer to FIGS. 8A and 8B). The HRTEM image of GQD-Cur shows a round-shaped Cur-encapsulated GQD composite, which is relatively larger than GQD, with an average size of about 100 nm, as shown in image (g) of FIG. 3.

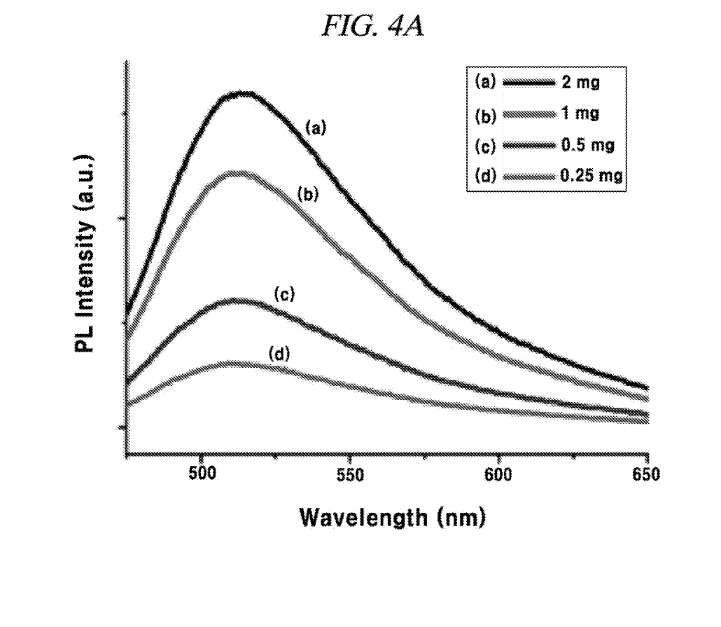

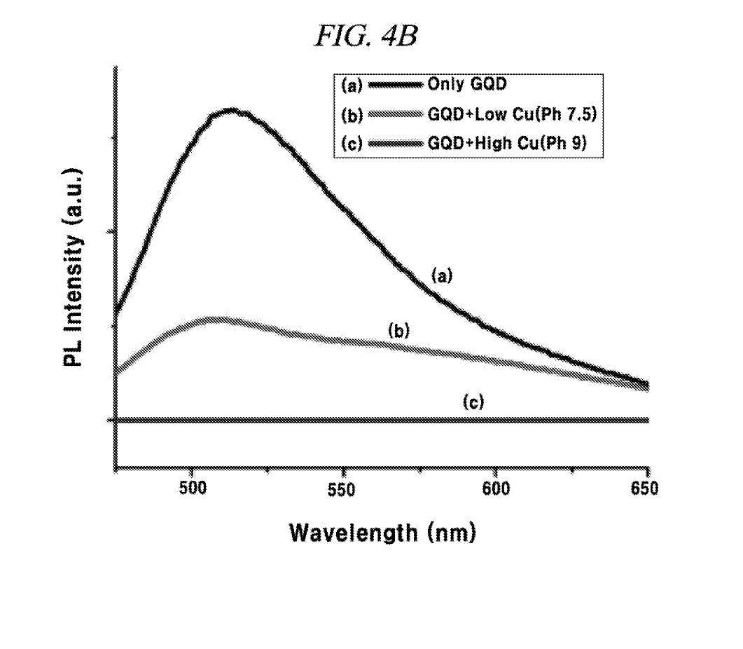

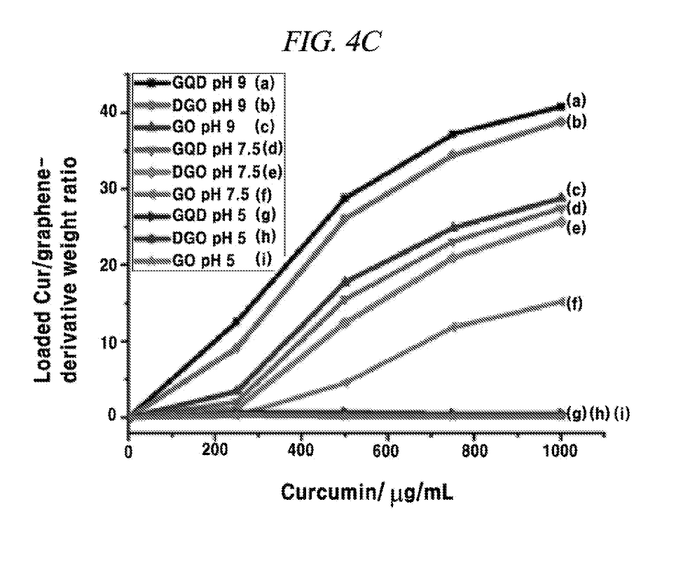

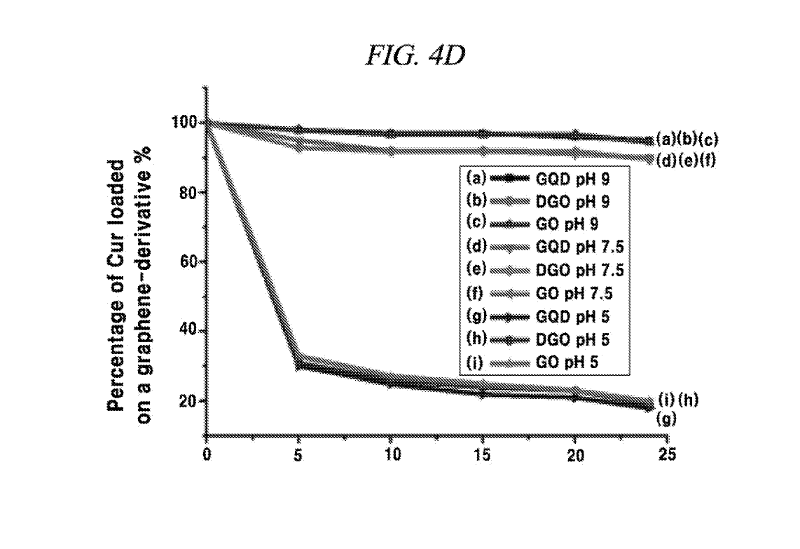

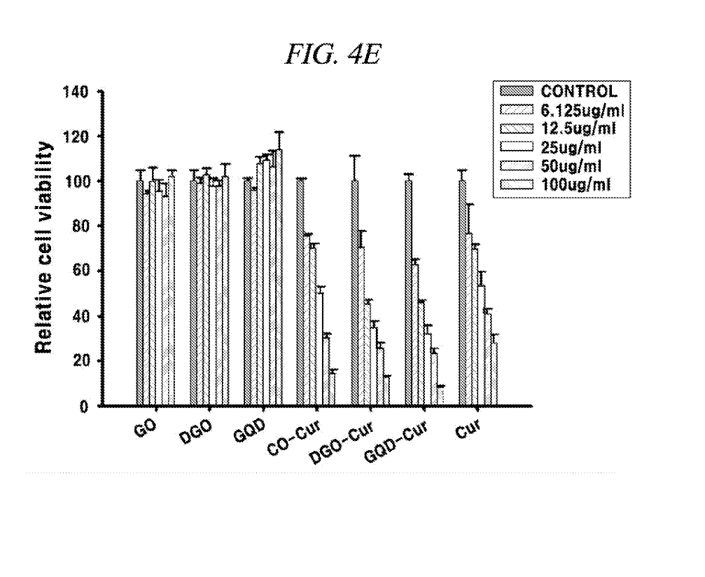

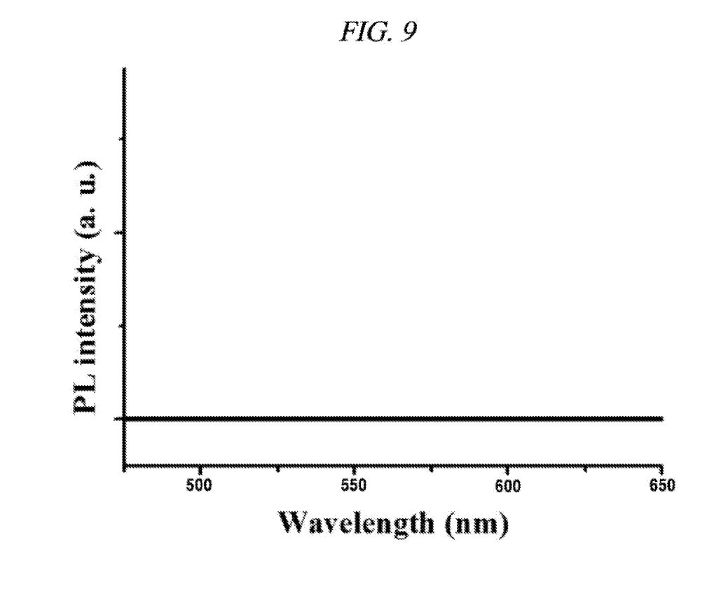

GQD exhibited an excellent photoluminescence (PL) image owing to UV excitation (400 nm). FIG. 4A shows photoluminescence (PL) intensity of GQDs, indicating concentration-dependent PL intensity of GQDs at an emission wavelength of 512 nm. However, GQD formed the composite with Cur, and relative PL of the composite gradually decreased with an increasing amount of Cur (FIG. 4B), whereas Cur alone had no PL intensity (refer to FIG. 9).

With respect to conventionally reported PL intensity, sufficient fluorescence intensity of GQDs was maintained for 3 days due to their low stability in an aqueous solution. Moreover, GQD exhibits a pH-dependent PL behavior; PL intensity decreases in an aqueous solution with high or low pH.

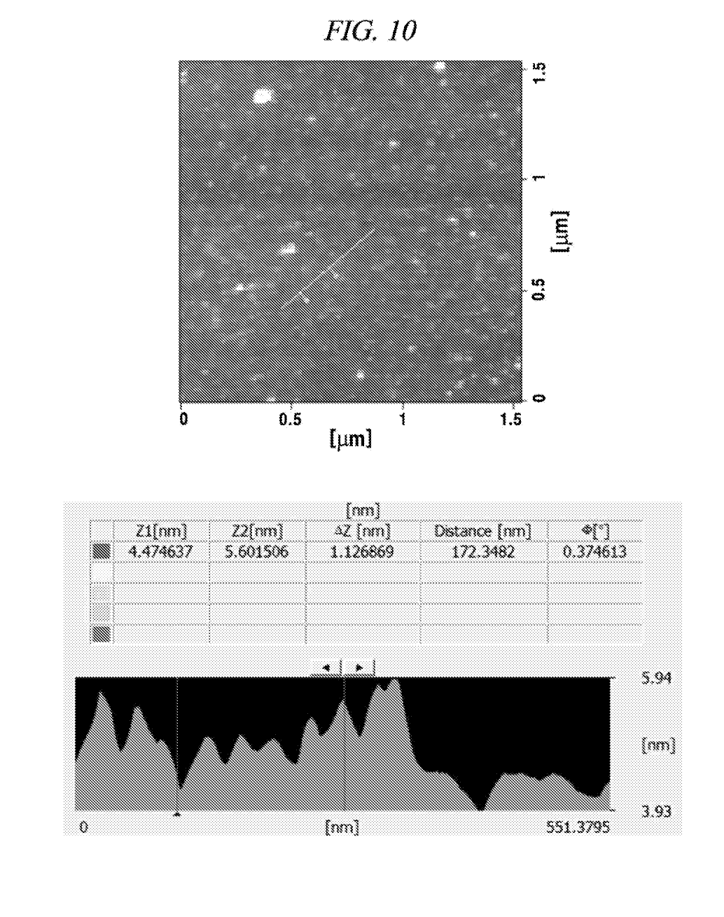

The cross-sectional view of the AFM image shows about 1 nm topographic height, which is an obvious example for a single layer of GQDs (refer to FIG. 10). The chemically synthesized GQDs can be easily dispersed in water due to the oxygen-containing functional groups, which was confirmed by FT-IR measurement. The free-standing graphene derivatives (the above-prepared GO, DGO, and GQD) at a concentration of 200 μg/mL were mixed with 1 mg Cur in an alkaline aqueous solution (about pH 9) under sufficient stirring.

The mixed aqueous solution became initially turbid because of poor aqueous dispersibility of pure Curcumin, but the mixed solution was gradually dispersed within a few minutes like the Cur nano-composite and gradually dispersed within water as the Cur nano-particles were increasingly absorbed to the graphene derivative surfaces through interaction between the Cur molecules and the alkaline graphene derivative surfaces. Clean aqueous solution was finally observed because a large amount of Cur nano-particles were loaded on the graphene derivatives, and thereby, forming GO-Cur, DGO-Cur and GQD-Cur composites. Centrifugation was carried out to remove residual Cur molecules that were not loaded on the graphene derivatives.

In sum, the above-prepared GO-Cur and DGO-Cur composites were precipitated under centrifugation (14,000 rpm, 20 minutes), while free Cur molecules remained in the supernatant because of their low molecular weights. Thereafter, the precipitate was collected and washed with DI water several times, while the GQD-Cur composite was collected by recrystallization of unattached Cur and evaporation of the resulting aqueous solution.

### Example 2

### Experiment of pH-Dependence of the Graphene Derivative-Based Composition for Drug Delivery

The loading behaviors of Cur on the graphene derivatives in an acidic to alkaline environment covering a range of pH 5 to pH 9 were quantitatively studied. The concentration of loaded Cur was determined by the calculation of free Cur (FIG. 4C). The present disclosure discovered that the amount of Cur bound to the graphene derivatives was pH-dependent.

The loading factors (defined as the graphene derivative/Cur weight ratio) were about 40.8, about 38.8 and about 20.8 for GQD-Cur, DGO-Cur and GO-Cur, respectively (FIG. 4C). In all the cases, the amount of loaded Cur gradually decreased from a large amount to a very small amount as pH was reduced from 9 to 7.5, and then, to 5 (FIG. 4D). This tendency was attributed to protonation, and as a result, the interaction between Cur and the graphene derivatives was reduced. A similar type of pH-dependent loading of nanomaterial-based nano-carriers has been conventionally reported. Further, the Cur-loading efficiency for different initial Cur concentrations in the same concentration (200 μg/mL) of the graphene derivatives has been investigated.

As shown in FIG. 4C, the amount of Cur-loading on the graphene derivatives gradually increased with an increasing initial Cur concentration in neutral and alkaline environments (pH 7.5 to pH 9).

Of particular significance was that the loading capacity of Cur rapidly increased to 40,800 mg/g under optimal conditions for GQD (e.g., pH 9 and 1 mg Cur). Based on the experimental data by the inventors of the present disclosure, it can be concluded that GQD has a larger amount of Cur nano-particles than that of GO or DGO.

Next, Cur-release behaviors of the composites prepared at pH 9 was investigated. The concentration of released Cur is determined by measuring free Cur. In particular, Cur molecules stacked on the graphene derivatives stably remained in a buffer close to alkaline and neutral, and about 9.8% or about 5% Cur in the buffer was released from the graphene derivatives at pH 7.5 or pH 9 in 24 hours.

In sharp contrast, Cur was released as much as about 85% from the graphene derivatives at pH 5 for 24 hours (FIG. 4D), due to the protonation and the subsequent reduced interaction between Cur and the graphene derivatives in the acidic environment. It is well-known that the pH-dependent drug-loading and the releasing property are favorable for cancer therapy, and this is because the microenvironment of extracellular tumor tissues and intracellular lysosomes and endosomes is acidic, whereby active drug is promoted to be released from the above-prepared composite materials.

### Example 3

### Evaluation of Cytotoxicity of the Graphene Derivative-Based Composition for Drug Delivery

In order to evaluate and compare in vitro cytotoxicity of the composites, GO, DGO, GQD, Cur, and Cur loaded on GO, DGO, and GQD, cell viability was measured by using a typical cancer cell line, i.e., HCT116 (human colon adenocarcinoma cell). As expected, the cell viability was 90% or more for GO, DGO, and GQD, which verified that the graphene derivatives have biocompatibility (FIG. 4E).

As shown in FIG. 4E, about 90% of the cells were dead by the Cur composite at the concentration of 100 μg/mL. Of all the composites, GQD-Cur was the most effective with death of 90% or more of the cancer cells, and less than 90% of the cancer cells were dead in GO-Cur and DGO-Cur. The death rate of the cancer cells in case of using Cur only was about 70%, which is a low value in the same condition. In addition, the GQD-Cur composite was very effective as 40% of the cells were dead in a very low concentration (6.125 μg/mL). It can be explained that the high cytotoxicity of the composite molecules, compared to using Cur alone, is attributed to the large surface area of GO, DGO, and GQD, which enables polar interaction with the oxygen-containing functional groups, and accordingly, two (2) essential factors for drug-loading in the composites can be improved.

In addition, Cur of the composites formed small-sized (0.15 μm to 0.1 μm) nanoparticles, compared to using Cur alone (about 1 μm) (FIG. 3D), which also increases the reaction sites. Compared to the high cytotoxicity of the graphene derivative-Cur composite, the cells incubated by using the pure graphene derivative maintained high cell viability (>90%), suggesting that the graphene derivative can be used as a drug nano-carrier having no cytotoxicity owing to the favorable biocompatibility of graphene.

### Example 4

### Evaluation of Cell Viability of the Graphene Derivative-Based Composition for Drug Delivery

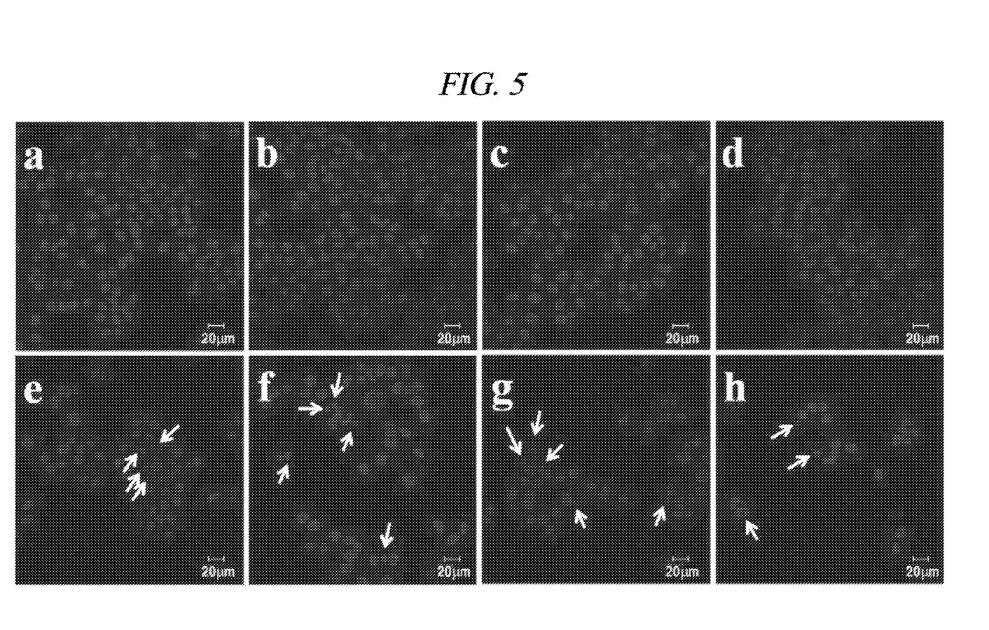

For further evaluation of the cell viability, the cells were stained with 4,6-diamidine-2-phenylindole dihydrochloride (DAPI), and observed by a fluorescence microscope (FIG. 5A to FIG. 5H). As a result, a synergistic effect was achieved in the system of the present disclosure. It was observed that the treatment with the GQD-Cur and DGO-Cur composites increased production of condensed and/or fragmented nuclear and the percentage of hypodiploid cell population in HCT116, indicating apoptotic cell death, compared to cells treated with GO-Cur or Cur alone (FIG. 5). In images (e) and (h) of FIG. 5, the condensed and/or fragmented nuclear is marked by arrows, providing evidence of the apoptotic cell death. Thus, the Cur molecules were efficiently released from the composites distributed within the cells due to the acidic condition (pH 5). As a result, the graphene derivative-Cur composites accumulated within the cells enable continuous Cur release, which ensures Cur accumulation and a sufficient drug concentration within the cells so as to continuously kill cancer cells.

### Example 5

### Study of an In Vivo Therapy Effect of the Graphene Derivative-Based Composition for Drug Delivery

A remarkable synergistic effect in vitro using the DGO-Cur and GQD-Cur composites, compared to when Cur alone exists, and subsequently, an in vitro therapy effect of the same composites for mice with HCT tumors transplanted into their back were investigated.

Experimental female nude mice bearing subcutaneous xenografts were divided into groups and intratumorally injected with a single dose of each of physiological saline, pure DGO and GQDs, free Cur, or DGO-Cur and GQD-Cur composites. The present experiments used six (6) groups of tumor-transplanted mice, each of which includes six (6) mice. For mice injected with Cur or the graphene derivative-Cur nano-composites, a concentration of 10 mg/kg was selected. As expected, the graphene derivative-based nano-composites in the tumor sites contribute to enhancing the tumor therapy effect, and this is because the stable and continuous release of Cur from the Cur-graphene composites can effectively kill cancer cells and inhibit tumor proliferation.

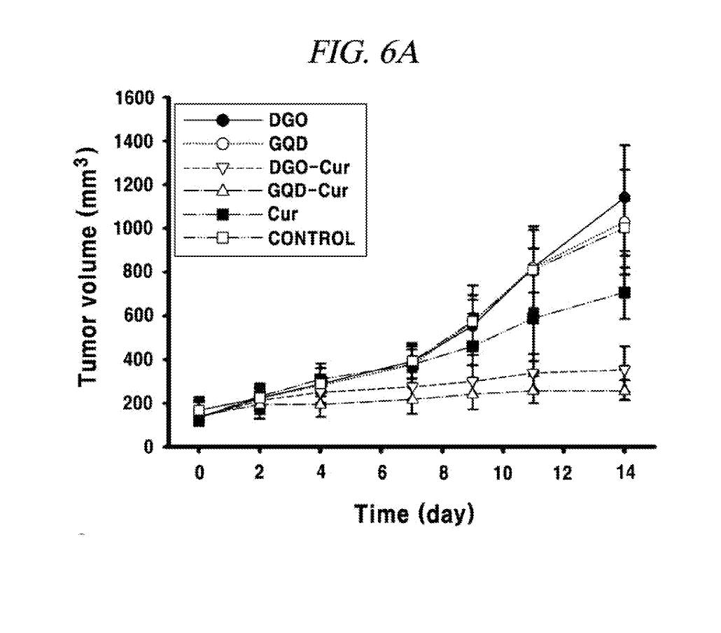

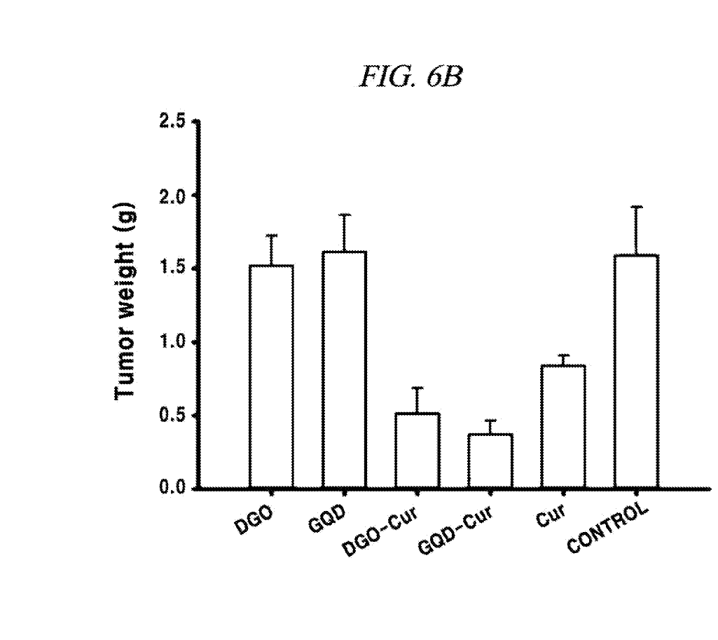

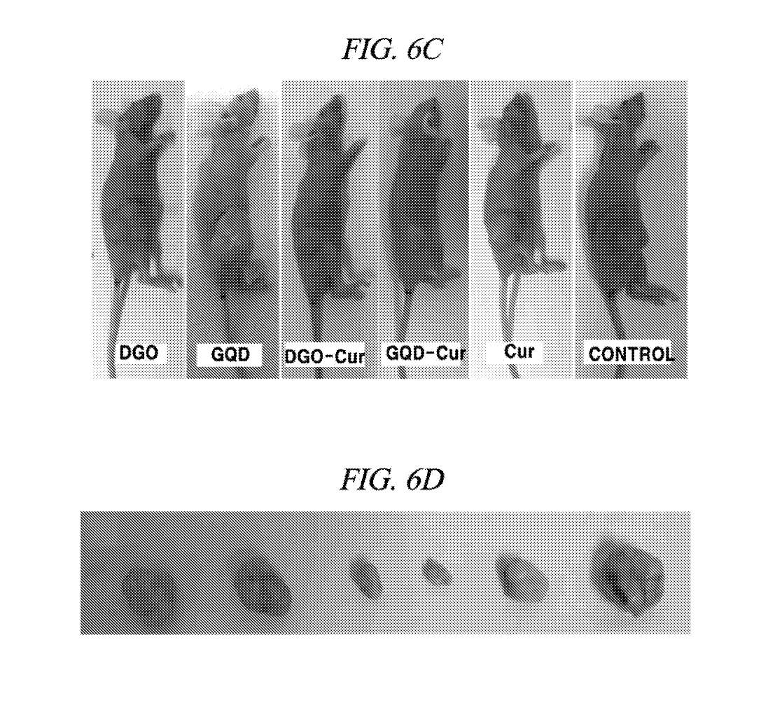

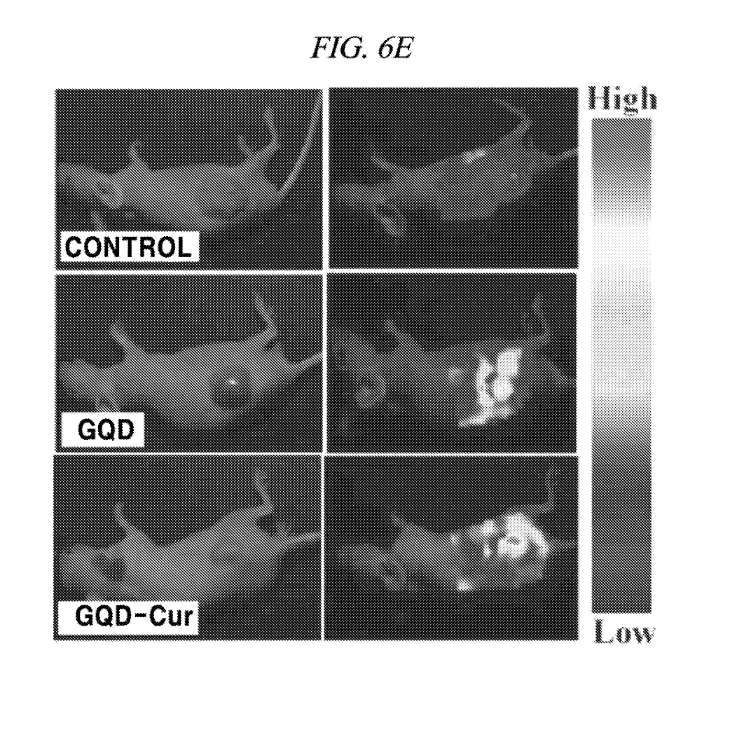

Quantitative measurement of the tumor proliferation inhibition was analyzed by detection of a tumor proliferation rate in terms of tumor volume change, further verifying the superior therapeutic effect of the graphene derivative-based nano-composites. FIG. 6A shows the tumor volume measured for each of the groups according to time. The graph of FIG. 6A shows increase in tumor volume of three (3) control groups with increasing time, namely, average tumor volumes (V, mm³) of the mice injected with PBS, GQD, or DGO were about 1,000, about 1,027, or about 1,100 for 14 days, respectively. On the other hand, in case of another control group of mice treated with free Cur, tumor proliferation was initially inhibited to some extent, and a tumor size increased as expected (FIG. 6A).

In contrast, the DGO-Cur and GQD-Cur groups exhibited remarkable inhibition of tumor proliferation, namely, the mice treated with DGO-Cur and GQD-Cur survived for 14 days with almost no tumor proliferation observed, and this result is comparable with any other reported processes for tumor proliferation using a nanomaterial-based drug delivery composition for cancer therapy. According to the in vivo results, the GQD-Cur composite was more effective than the DGO-Cur composite.

### Example 6

### Study of Non-Invasive Optical Imaging of the Graphene Derivative-Based Composition for Drug Delivery

After injection of GQD or the GQD-Cur composite into mice at 10 mg/kg, intratumoral GQD distribution was analyzed by non-invasive imaging of GQDs in the mice by using an Optix in vivo imaging system (Optix MX3, ART Advanced Research Technologies INC, Canada). As shown in FIG. 6E, no GQD fluorescence was observed in the mice treated with PBS (control sample). In contrast, distinct GQD fluorescence signals were observed in the tumors injected with GQD and GQD-Cur, respectively. However, after release of Cur from the composites, remaining GQD had fluorescence signals, but the GQD-Cur nano-composite had no fluorescence signal.

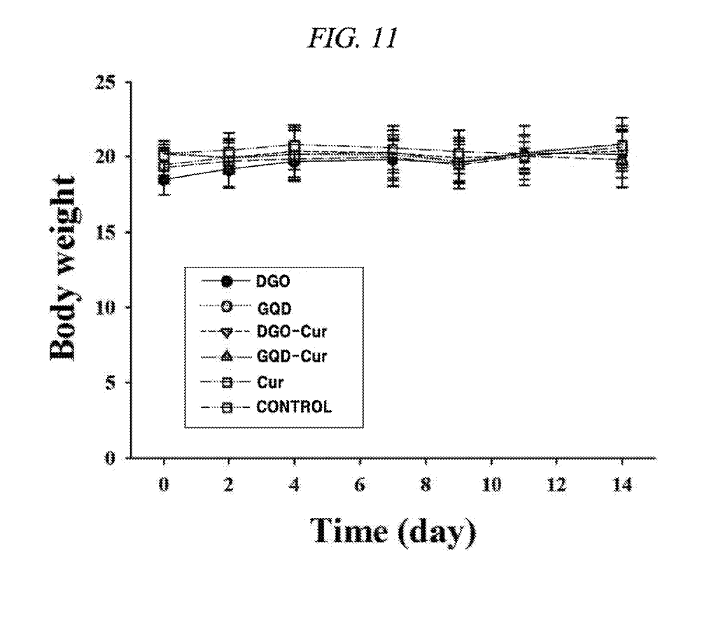

These results verify that the GQD nano-composite can be used for cancer therapy, and simultaneously, can be used as a bioprobe for tumor imaging. According to conventional reports, nano-sized GQDs were accumulated in tumors through a reticule endothelial system (RES), and since GQDs are not target-specific, the fluorescence gradually decreased as blood circulated. With respect to the excellent synergistic therapy effect of the graphene nano-composite system, long-term retention and in vivo toxicity should be specifically investigated prior to use in medical applications. The present disclosure did not detect either death or significant weight reduction, which was not observed in all the groups tested (refer to FIG. 11).

### Example 7

### Histological Analysis of the Graphene Derivative-Based Composition for Drug Delivery

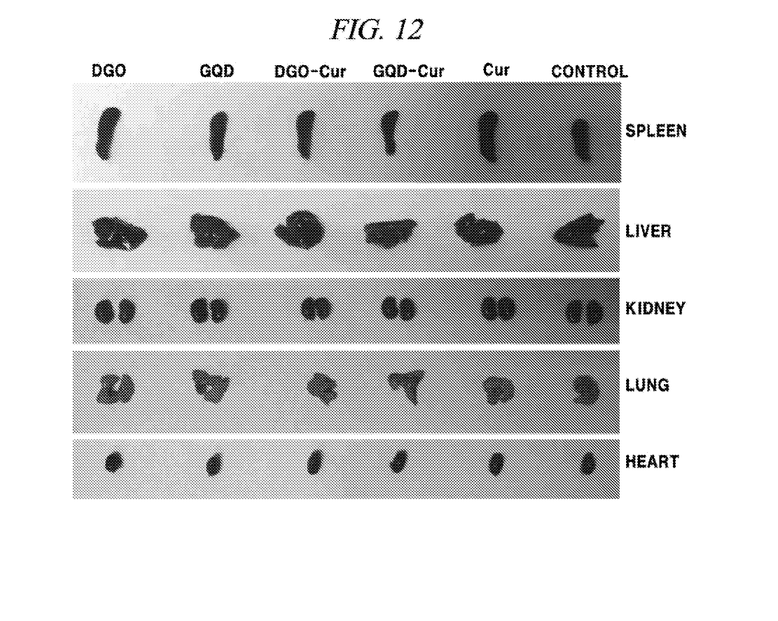

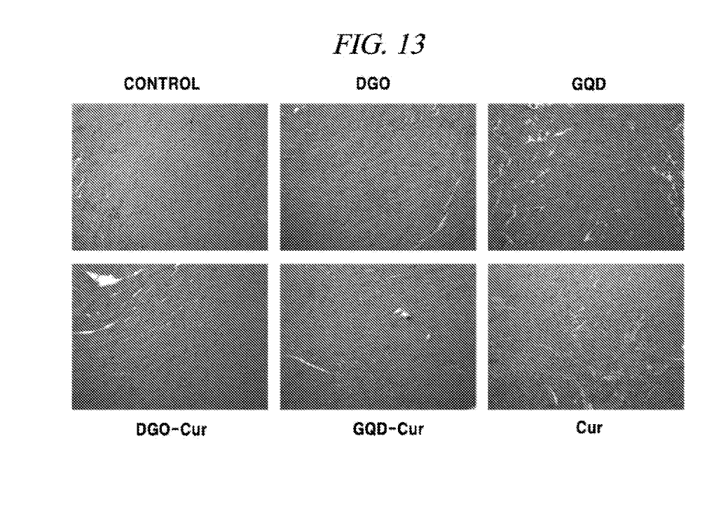

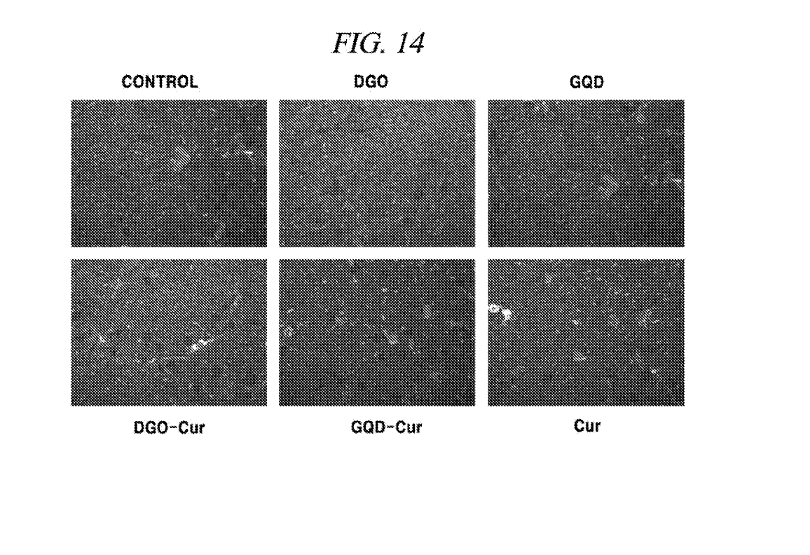

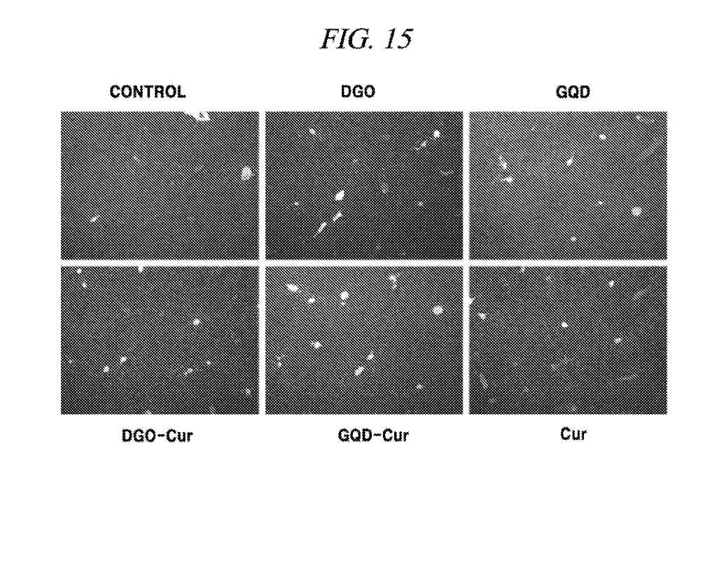

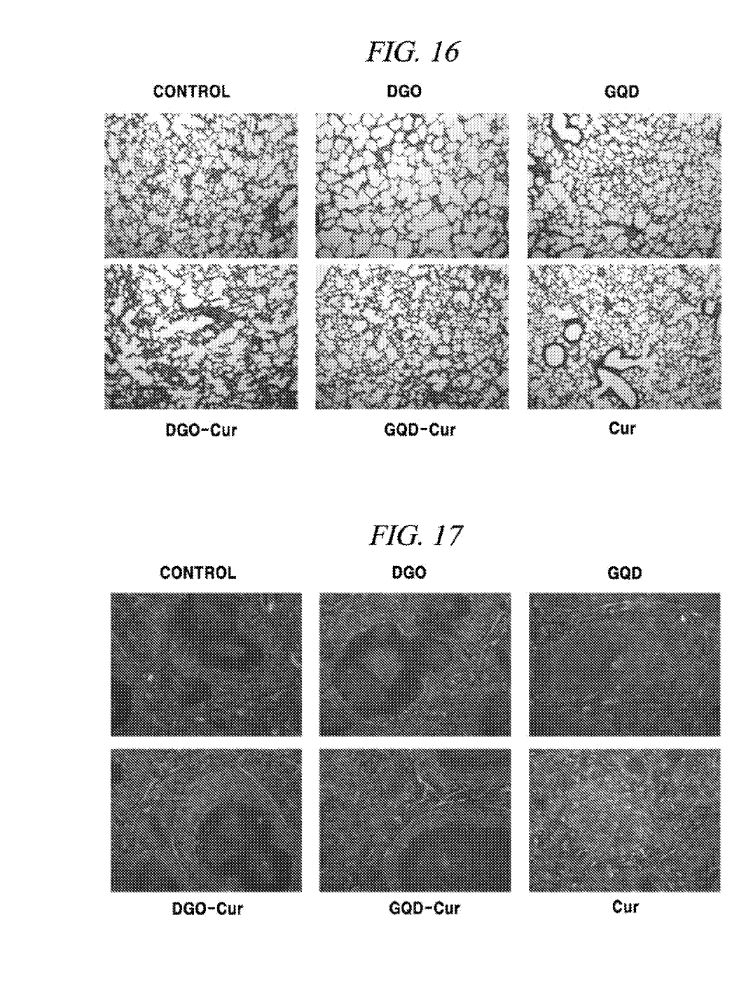

Long-term toxicity was detected by observing histological changes of the most essential organs, such as liver, spleen, kidney, heart, and lung. There were no histological lesions or any other negative effects in association with the injection of the nanomaterials of the present disclosure (FIG. 13 to FIG. 17). On the other hand, the structure of the most essential organs of the exposed mice was normal like those of the control group (refer to FIG. 12). These results indicate that the graphene derivative-Cur nanoparticles serve as promising materials for synergistic or stable medicine.

To summarize the present disclosure, the present disclosure shows that the graphene-based nano-carrier can be used as part of a high-performance hydrophobic drug-delivery platform for delivery of the anticancer drug Cur. Of particular significance, the graphene derivative-Cur composites have the property of extremely large Cur-loading capacity (40,800 mg/g), which is higher than that of conventionally reported nanomaterial-based drug deliveries. Among the other graphene derivatives, GQD can produce the composites and deliver Cur drug in a large amount.

The in vitro experiments show that the graphene derivative-Cur composites enable Cur release and effective cancer cell destruction. The present disclosure has demonstrated that the Cur-loaded graphene derivatives are highly effective in tumor growth inhibition as the mice treated with GQD-Cur or DGO-Cur survived for 14 days without any detectable tumor proliferation.

In addition, the present disclosure has demonstrated the synergistic effect in cancer cell viability both in vitro and in vivo; the graphene derivative-Cur nano-composite system has the highest anticancer activity among all the GQD-Cur composites. As the most excellent investigation, the present disclosure is the first example for synergistic chemotherapy of cancer cells in vitro and vivo, simultaneous with a superficial bioprobe for tumor imaging, using the GQD-Cur composites.

Considering that the graphene derivatives can be easily prepared with relatively high yield and low costs, the graphene-based nano-deliveries are realizable strong means for cancer therapy, and can be used as materials competing with or complementing nanomaterial-based nano-deliveries in the art of the present disclosure.

With the superior stability, high biocompatibility, and excellent synergistic therapy effect, the drug-delivery system will be a promising in vivo cancer therapy agent, verifying increasing use in biological and medical applications through additional modification.

Our knowledge of the biological properties of Cur and the graphene composites and their use in biomedical and biotechnological applications can be significantly advanced. Further, application of the composites to HIV infection, neurological, cardiovascular, and skin diseases are being investigated.

While this disclosure includes specific examples, it will be apparent to one of ordinary skill in the art that various changes in form and details may be made in these examples without departing from the spirit and scope of the claims and their equivalents. The examples described herein are to be considered in a descriptive sense only, and not for purposes of limitation. Descriptions of features or aspects in each example are to be considered as being applicable to similar features or aspects in other examples. Suitable results may be achieved if the described techniques are performed in a different order, and/or if components in a described system, architecture, device, or circuit are combined in a different manner and/or replaced or supplemented by other components or their equivalents. Therefore, the scope of the disclosure is defined not by the detailed description, but by the claims and their equivalents, and all variations within the scope of the claims and their equivalents are to be construed as being included in the disclosure.

What is claimed is:

1. A graphene derivative-based composition, comprising:
a nanocomposite including a drug loaded on a carrier comprising a graphene derivative.

2. The composition of claim 1,
wherein the graphene derivative comprises a graphene oxide, a double-oxidized graphene oxide, or a graphene quantum dot.

3. The composition of claim 1,
wherein the drug comprises a drug for a treating or a preventing of a disease selected from the group consisting of a cancer, a HIV infection, and a neurological disease, a cardiovascular disease, and a skin disease.

4. The composition of claim 1,
wherein the drug comprises an antitumor agent.

5. The composition of claim 4,
wherein the antitumor agent comprises a curcumin, a doxorubicin, a paclitaxel, an etoposide, a vinca alkaloid, a vinblastine, or a colchicine.

6. The composition of claim 4,
wherein the drug has a nanoparticle shape.

7. The composition of claim 1,
wherein the graphene derivative has a relatively high content of oxygen-containing functional group.

8. The composition of claim 1,
wherein a released amount of the drug loaded on the carrier is controlled depending on a pH.

9. The composition of claim 8,
wherein the pH is controlled to an acidic, a neutral, and an alkaline range.

10. The composition of claim 1,
wherein a size of a particle of the nanocomposite ranges between about 50 nm and about 5,000 nm.

11. The composition of claim 1,
wherein the carrier comprises a graphene quantum dot, and the carrier is configured to deliver drug or to be used as a bioprobe for a cell imaging using a luminescent property of the graphene quantum dot.

12. A pharmaceutical composition comprising the graphene derivative-based composition of claim 1.

13. A method of preparing a graphene derivative-based composition, the method comprising:
adding a drug to a dispersion comprising a carrier comprising a graphene derivative to form a graphene derivative-drug nanocomposite in which the drug is loaded on the carrier.

14. The method of claim 13,
wherein the dispersion comprises water as a solvent.

15. The method of claim 13,
wherein a pH of the dispersion to which the carrier is added is controlled to an alkaline range.

16. The method of claim 13,
wherein the graphene derivative comprises a graphene oxide, a double oxidized graphene oxide, or a graphene quantum dot.

17. The method of claim 13,
wherein the drug comprises a drug for therapy or prevention of a disease selected from the group consisting of a cancer, a HIV infection, and a neurological disease, a cardiovascular disease, and a skin disease.

18. The method of claim 13,
wherein the antitumor agent comprises a curcumin, a doxorubicin, a paclitaxel, a etoposide, a vinca alkaloid, a vinblastine, or a colchicine.

19. The method of claim 18,
wherein the drug has a nanoparticle shape.

20. The method of claim 13,
wherein the graphene derivative has a relatively high content of oxygen-containing functional group.
